(19) United States
(12) Patent Application Publication
(10) Pub. No.: US 2003/0069599 A1
(43) Pub. Date: Apr. 10, 2003

(54) Device for holding an object by clamping it

(76) Inventor: Erich Tritt, Vorgaissweg (DE)

Correspondence Address: Gregory P. LaPointe, BACHMAN & LaPOINTE, P.C., Suite 1201, 900 Chapel Street, New Haven, CT, 06510-2802, US

(21) Appl. No.: 10/243,531

(22) Filed: Sep. 13, 2002

(30) Foreign Application Priority Data
Sep. 14, 2001 (DE) 101 45 401.5
Nov. 2, 2001 (DE) 101 53 868.5

(51) Int. Cl. A61B 17/28; A61B 17/42

(52) U.S. Cl. 606/205

(57) ABSTRACT

In a device for holding an object in surgery by clamping it, with a clamping jaw (36) which is provided at one end of a grip element (12) and which is limited by two mutually movable bits (32, 40), said bits (32, 40) are in each case the endpiece of a profile element (30, 44); the profile elements are connected to one another in one piece by at least one integrally formed spring member (46), and the clamping jaw (36) is actuated by the relative movement of the profile elements. One of the profile elements (30) is connected to the grip element (12) in a fixed manner, and the other profile element (44) is attached to the first profile element (30) by means of the spring member (46) and is mounted pivotably thereon.

The invention relates to a device for holding an object in surgery by clamping it, with a clamping jaw which is provided at one end of a grip element and which is limited by two mutually movable bits.

Devices of this kind are generally known as forceps, needle holders, scissors or tongs. Thus, for example, document DE 82 06 782 U1 from the Applicant discloses scissors with round grip for microsurgical purposes, which are intended to be used in particular in microvascular anastomoses and neurotizations and in which a cylindrical grip part is divided into two semicylindrical branches by a slit along its longitudinal axis and along part of its length; the free ends are configured as branches of scissors with cutting edges and are connected to one another in a pivotable manner at a distance from their ends by a bearing pin. In addition, with a view to forming a spring part, the semicylindrical branches at the point of transition to the scissor branches are intended to be tapered except for a blade-like connection portion on both sides of the slit.

In view of this prior art, the inventor has set himself the object both of simplifying production and of improving handling and cleaning after use.

The teaching of independent claim 1 achieves this object. The dependent claims specify favorable refinements. In addition, all combinations of at least two of the features disclosed in the description, the drawing and/or the claims fall within the scope of the invention.

According to the invention, the bits are in each case the endpiece of a profile element, and these profile elements are connected to another by at least one integrally formed spring member; the clamping jaw can be actuated by the relative movement of the profile elements.

It has proven expedient to connect one of the profile elements to the grip element in a fixed manner and to attach the other profile element to the first profile element by means of the spring member(s) and to mount it pivotably thereon; by virtue of this measure, an insert part can be made up from the two profile elements (connected by the spring(s)) and their bits and they can be connected jointly to the grip element. This insert part is intended to have a flat configuration, its thickness corresponding to a fraction of the width or diameter of the grip element so as to be able to produce a slit connection.

According to a further feature of the invention, the mutually facing inner edges of the bits can be moved onto one another or moved away from one another upon a pivoting movement of the profile element on the spring member(s). For this purpose, it is advantageous for the movably mounted profile element to be provided, at a distance from its bit, with a press surface or similar touch element, the press surface or similar touch element preferably being arranged on that end of the profile element remote from the bit, in order to obtain the greatest possible lever arm upon actuation.

It has proven expedient for the press surface of the movably mounted profile element to be provided on a profile portion which engages through a recess of the other profile element or of the grip element; the profile portion should preferably be bent out from the profile element toward the longitudinal axis.

In this configuration, a single spring member is integrally formed at both ends of the profile elements near the bits and crosses the longitudinal axis of the device. In this embodiment according to the invention, the grip element is preferably of tubular configuration and is provided at one end with an axis-parallel crest slit or ridge slit for receiving the rod-shaped or plate-shaped profile element of the above-mentioned insert part which can be secured therein; the mutually facing bits should protrude beyond the front edge of the grip element. The width of the crest slit or ridge slit corresponds to the thickness of the insert part, so that the latter can be inserted into said slit.

According to another feature of the invention, the movably mounted profile element engages radially through a keel slit of the grip element and is movable in it; this keel slit lies parallel to the crest slit and, with the latter, in an axial plane of the grip element.

A ridge recess of the grip element for the profile portion containing the press surface is also intended to be assigned to the keel slit at its end remote from the bits, on the other side of the longitudinal axis; the width of the ridge recess is chosen to be greater than the width of the axially adjoining ridge slit.

In a further embodiment according to the invention, provision is made for a pair of spring members which are formed integrally at a distance from one another between the profile elements, the spring member integrally formed at both ends preferably being of rod-shaped or plate-shaped configuration.

This grip element advantageously has, at one front end, an approximately axis-parallel jib to which one profile element is attached, the latter being connected to the other profile element of an insert part by means of a pair of spring members. One of the profile elements is in this case welded to the front face of the jib, i.e. connected to it by adherence.

It has proven expedient for the movable profile element to be provided, in each rod-shaped portion extending between the spring members, with a resilient intermediate element, in which case a press arm with press surface protrudes beyond the spring member remote from the bits. That spring member is intended to lie in the area of a depression or valley of the rod-shaped portion in the movable profile element, i.e. to be a part of the latter; when pressure is applied to the press arm, it serves to generate a bending of the profile element and thereby to actuate the clamping jaw.

Finally, protection is claimed for a design in which one of the rod-shaped or plate-shaped spring members has a middle portion bent in a U-shape; the grip element is in this case connected at the front to a profile element in a fixed manner, and the movable profile element is joined to the fixed profile element by a rod-shaped spring member and, at a distance therefrom, by that spring member provided with the middle portion of U-shaped cross section. This folded spring member can also serve as a kind of block against twisting; with it, a further guide member would be superfluous.

That spring member with the middle portion of U-shaped cross section is intended to be assigned to the press surface of the movable profile element which engages through a ridge recess or similar aperture of the grip element.

The scope of the invention moreover covers a device which replaces a joint in a two-dimensional plane and can be used for all plane-related movements such as holding, gripping, cutting, positioning or the like. Moreover, it is intended to comprise an integrated spring, so that the working part returns to the starting position ideally without friction or external forces. A device of this kind can also be used outside the field of instrument technology, for example in robots.

All in all, a novel clamping device is provided which effectively achieves the object set out in the introduction.

Further advantages, features and details of the invention will become evident from the following description of preferred illustrative embodiments and by reference to the drawing, in which:

FIGS. 1, 6, 8 each show a part of surgical pincers or forceps in their rest position, in partially cutaway side views;

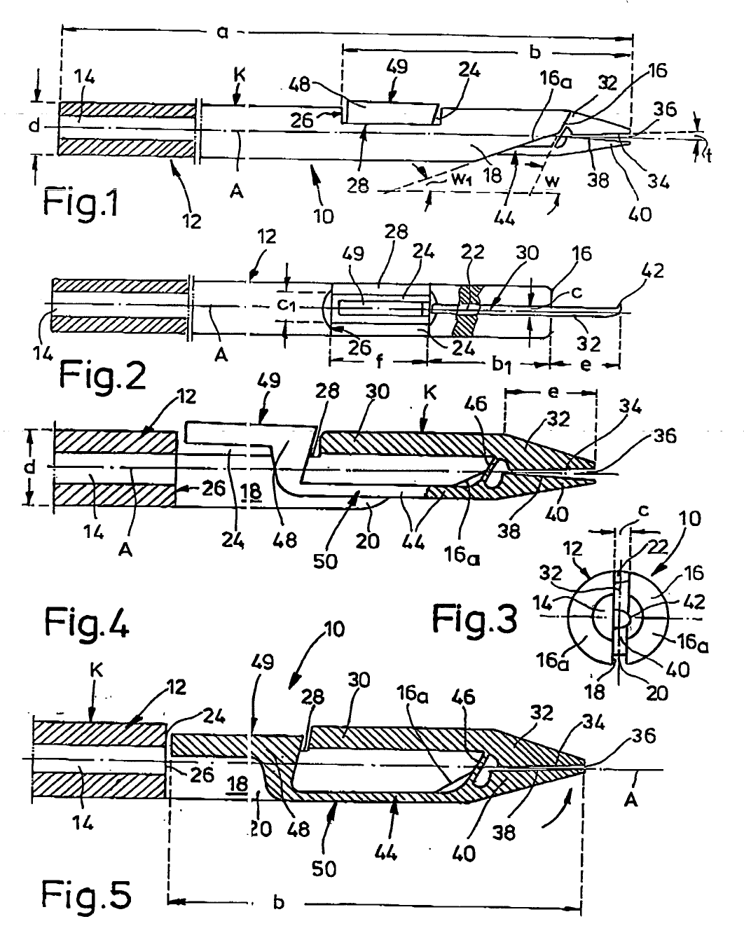

FIG. 2 shows a plan view of FIG. 1;

FIG. 3 shows an enlarged front view of FIG. 1;

FIG. 4 shows an enlarged partial longitudinal section through FIG. 1;

FIG. 5 shows the view according to FIG. 4 in the gripping position;

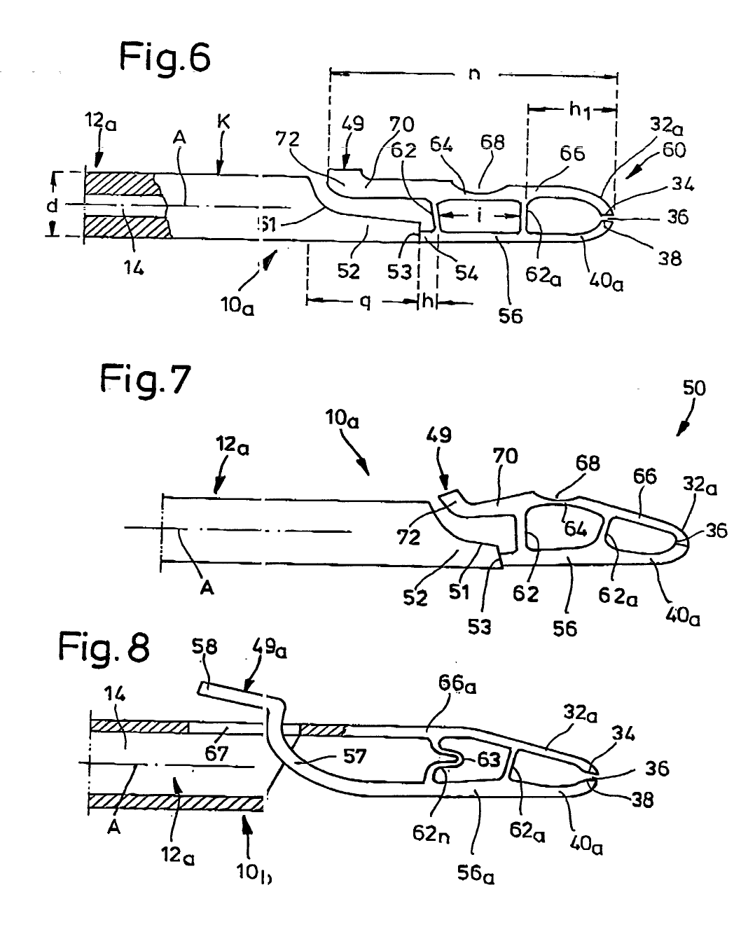

FIG. 7 shows the device according to FIG. 6 in the gripping position.

A pincer device 10 or forceps with length a of approximately 150 mm has an in this case cylindrical grip shaft 12 of diameter d, in this case for example 10 mm, with a longitudinal bore 14 extending in the longitudinal axis A.

The right-hand front edge 16 of the grip shaft 12 shown in FIG. 1 is, in an upper area, inclined at an angle w of about 70° to the longitudinal axis A and, in the remaining part, at an angle w₁ of approximately 25°, to form two inclined edges 16ₐ. These two inclined edges 16ₐ delimit two cross-sectional branches 18 of the grip shaft 12 which in turn flank an axis-parallel keel slit 20, with length b of 40 mm, starting from the longitudinal bore 14.

Lying opposite the lower keel slit 20, on the other side of the longitudinal axis A, there is a crest slit 22 of length b₁, in this case 20 mm, and of width c, in this case approximately 2 mm, passing through the grip shaft 12. Axially adjoining the crest slit or ridge slit 22 there is a ridge recess 24 which extends over the whole width of the grip shaft 12 and, with the keel slit 20, has a common rear wall 26 as upper surface. The ridge recess 24 in the grip shaft 12 defines two flank edges 28 which extend almost radially to the longitudinal axis A. A metal strip 30 is inserted into and secured in the axis-parallel crest slit or ridge slit 22 of the grip shaft 12, seen in particular in FIG. 2, which metal strip 30, with its free end designed as bit 32 (triangular in side view), protrudes axially beyond the front edge 16 by a distance e of 15 mm. In the rest position indicated in FIG. 1, the inner edge 34 of the bit 32 extends with respect to the adjacent longitudinal axis A at a low angle t, in this case of less than 10°. It defines one side of a clamping jaw 36 which opens out at the end and whose other side is formed by the counter-inclined inner edge 38 of a lower and likewise triangular bit 40.

It will be seen in particular from FIGS. 2 and 3 that, on the one hand, the free pointed ends 42 of the bits 34, 40 are curved radially out from their bit planes and, on the other hand, that the lower bit 40 is part of a metal strand 44 where, at the transition of the upper bit 34 with the front edge 16, a spring strip 46 is held on the grip shaft 12, which spring strip 46 is formed integrally on the upper metal strip 30 and is likewise in one piece with the other metal strand 44; the longitudinal axis A of the grip shaft 12 is crossed almost radially by this spring strip 46. The metal strips 30, 44 connected in this way by the spring strip 46 thus belong, together with their bits 34, 40 (free length e of about 15 mm) to a one-piece insert part 50 with total length b of approximately 55 mm.

The insert part 50 also comprises a contact strip 48 (of approximately L-shaped configuration in the side view in FIGS. 4 and 5) which is formed integrally on the lower metal strip 44 and whose surface 49 serves as the press surface in order to guide said inner edges 34, 38 of the bits 32, 40 toward one another counter to the force of the spring strip 46. The contact strip 48 is mounted movably in that ridge recess 42 (with length f in this case of approximately 20 mm) of the grip shaft 12 whose flank edges 28 from the grip piece 12 are, as has been stated, flush with one another as side walls in one plane. The width c₁ of the ridge recess 42 is greater than the width c of the adjoining ridge slit 22.

In the rest position according to FIGS. 1 and 4, the clamping jaw 36 is open and the contact strip 48 protrudes slightly above the ridge contour K of the grip piece 12; in the gripping position according to FIG. 5, the inner edges 34, 38 of the bits 34, 40 lie on one another, as has been stated, and the press surface 49 is lowered into the ridge recess 42.

In the embodiment of the pincer device or forceps 10ₐ according to FIGS. 6 and 7, a forceps-like gripping element 60 with free length n of approximately 50 mm and with a clamping or gripping jaw 36 at the end, is welded at the end of the grip shaft 12 at 54 onto the front face 53 of a jib 52 of jib length q of the grip shaft 12a whose cross section has the shape of a segment of a circle, which is integrally formed in a projecting manner on the grip shaft 12 and lies parallel to and below the longitudinal axis A. Said clamping or gripping jaw 36 is delimited by two metal strips which serve as bits 32a, 40a and are bent in an arc of a circle in longitudinal cross section.

This gripping element 60 has a lower rod-shaped profile element 56 which issues rigidly from the weld point 54 and from which a spring rod 62, 62ₐ extends upward, on the one hand at a short distance h from the weld point 54 and, on the other hand, and at a distance h₁ from the gripping jaw 36; the clear distance of these two spring rods 62, 62ₐ from one another is indicated by i, and, at their upper ends, these spring rods 62, 62ₐ are connected in one piece to a comb-shaped profile rod 66.

Between the two spring rods 62, 62ₐ crossing the longitudinal axis A, a valley 68 can be seen in the upper profile rod 66. This valley is delimited from underneath by a further spring element 64 which extends approximately parallel to the axis. Extending from the first-mentioned rearward spring rod 62, a portion of the upper profile rod 66, free at one end, extends above the jib 52 (delimited by a surface 51 of curved longitudinal cross section) of the grip shaft 12a and, in FIG. 6, parallel to the latter, and it forms a press arm 70 which, on a radially upwardly projecting arm piece 72, provides short press surface 49.

If pressure is applied to the press surface 49 of the press arm 70, the upper profile rod 66 curves upward at and by virtue of its spring element 64 delimiting the valley 68, and the upper bit 32ₐ is pushed onto the lower bit 40ₐ of the clamping or gripping jaw 36. FIG. 7 shows the described gripping jaw 36 in the closed position which, as has been stated, is obtained by pressure on the press arm 70. Here, therefore, the three spring elements 62, 62ₐ, 64 control the corresponding gripping movement of the clamping or gripping jaw 36.

In the device 10_{b} in FIG. 8, the rear spring rod 62ₙ is folded in the area of a middle portion 63 of U-shaped longitudinal section and is configured as a restoring spring; the front spring rod 62ₐ is very thin and is only loaded by traction. The rear spring rod 62ₙ also represents a kind of barrier against twisting.

In this case, the lower profile element 56ₐ is bent upward behind the folded spring rod 62ₙ and engages with its curved arc portion 57 through an aperture 67 in the upper profile element 66ₐ. A grip end 58 bent out from the surface of the arc portion 57 protrudes outside the upper profile element 66ₐ and provides a press surface 49ₐ. In this device 10_{b}, the clear height of the working end can be made much smaller.

1. A device for holding an object in surgery by clamping it, with a clamping jaw (36) which is provided at one end of a grip element (12, 12ₐ) and which is delimited by two mutually movable bits (32, 40; 32ₐ, 40ₐ), wherein the bits (32, 40; 32ₐ, 40ₐ) are in each case the endpiece of a profile element (30, 44; 56, 66; 56ₐ, 66ₐ), the profile elements being connected to one another in one piece by at least one integrally formed spring member (46; 62, 62ₐ, 62ₙ), and the clamping jaw (36) being actuated by the relative movement of the profile elements.

2. The device as claimed in claim 1, wherein one of the profile elements (30, 56, 66ₐ) is connected to the grip element (12, 12ₐ) in a fixed manner, and the other profile element (44, 66, 56ₐ) is attached to the first profile element by means of the spring member(s) (46; 62, 62ₐ, 62ₙ) and is mounted pivotably thereon.

3. The device as claimed in claim 1 or 2, wherein the two profile elements (30, 44; 56, 66; 56ₐ, 66ₐ) connected by the spring member(s) (46; 62, 62ₐ; 62ₙ) form with their bits (32, 40; 32ₐ; 40ₐ) an insert part (50) which is designed so that it can be connected to the grip element (12, 12ₐ), the insert part (50) having, if appropriate, a flat configuration and its thickness (c) corresponding to a fraction of the width or diameter (d) of the grip element (12, 12ₐ).

4. The device as claimed in one of claims 1 through 3, wherein mutually facing inner edges (34, 38) of the bits (32, 40; 32ₐ, 40ₐ) are arranged such that they can be moved onto one another or moved away from one another upon a pivoting movement of the profile element. (44, 56ₐ, 66) on the spring member(s) (46; 62, 62ₐ, 62ₙ).

5. The device as claimed in one of claims 1 through 4, wherein the movably mounted profile element (44, 66, 56ₐ) is provided, at a distance from its bit (32, 40; 32ₐ, 40ₐ), with a press surface (49, 49ₐ) or similar touch element, the press surface (49, 49ₐ) or similar touch element being arranged on that end of the profile element (44, 66; 56ₐ) remote from the bit (32, 40; 32ₐ; 40ₐ).

6. The device as claimed in one of claims 1 through 5, wherein the press surface (49, 49ₐ) of the movably mounted profile element (44, 66, 56ₐ) is provided on a profile portion (48, 58) which engages through a recess (24, 67) of the other profile element (30, 56; 66ₐ) or of the grip element (12, 12ₐ), the profile portion (48, 58) if appropriate being bent out from the profile element (44, 66, 56ₐ) toward the longitudinal axis (A).

7. The device as claimed in one of claims 1 through 6, having a spring member (46) which is integrally formed at both ends of the profile elements (30, 44) near the bits (32, 40) and crosses the longitudinal axis (A) of the device (10).

8. The device as claimed in one of claims 1 through 7, having a pair of spring members (62, 62ₐ; 62ₐ, 62ₙ) formed integrally at a distance (i) from one another between the profile elements (56, 66; 56ₐ, 66ₐ).

9. The device as claimed in one of claims 1 through 8, wherein the spring member (46; 62, 62ₐ) formed integrally at both ends has a rod-shaped or plate-shaped configuration.

10. The device as claimed in claim 8 or 9, wherein one of the rod-shaped or plate-shaped spring members (62ₙ) has a middle portion (63) bent in a U-shape. (FIG. 8)

11. The device as claimed in one of claims 1 through 7, wherein the grip element (12) is of tubular configuration and is provided at one end with an axis-parallel crest slit or ridge slit (22) for receiving the rod-shaped or plate-shaped profile element (30) of the insert part (50) which can be secured therein, the mutually facing bits (32, 40) protruding beyond the front edge (16) of the grip element. (FIG. 2)

12. The device as claimed in claim 11, wherein the width (c) of the crest slit or ridge slit (22) corresponds to the thickness of the insert part (50) and/or wherein the movably mounted profile element (44) engages radially movably through a keel slit (20) of the grip element (12) lying parallel to the crest slit (22) and, with the latter, in an axial plane of the grip element.

13. The device as claimed in claim 6 or 12, wherein a ridge recess (24) of the grip element (12) for the profile portion (48) containing the press surface (49) is assigned to the keel slit (20) at its end remote from the bits (22, 40), on the other side of the longitudinal axis (A), the width (c₁) of the ridge recess (24) optionally being greater than the width (c) of the axially adjoining ridge slit (22).

14. The device as claimed in one of claims 1 through 9, wherein the grip element (12ₐ) has at one front end an approximately axis-parallel jib (52) to which one profile element (56) is attached, the latter being connected to the other profile element (66) of an insert part (50) by means of a pair of spring members (62, 62ₐ).

15. The device as claimed in claim 14, wherein one profile element (56) is welded to the front face of the jib (52).

16. The device as claimed in claim 14 or 15, wherein the movable profile element (66) is provided, in its rod-shaped portion extending between the spring members (62, 62ₐ), with a resilient intermediate element (64), a press arm (70) with press surface (49) protruding beyond the spring member (62) remote from the bits (32a, 40ₐ).

17. The device as claimed in claim 16, wherein the spring member (64) is provided in the area of a depression or valley (68) of the rod-shaped portion in the movable profile element (66).

18. The device as claimed in one of claims 1 through 16, wherein the grip element (12ₐ) is connected at the front to a profile element (66ₐ) in a fixed manner, and the movable profile element (56ₐ) is joined to the fixed profile element by a rod-shaped spring member (62ₐ) and, at a distance therefrom, by the spring member (62ₙ) provided with the middle portion (63) of U-shaped cross section. (FIG. 8)

19. The device as claimed in claim 18, wherein the spring member (62ₐ) with the middle portion (63) of U-shaped cross section is assigned to the press surface (44ₐ) of the movable profile element (56ₐ) which engages through a ridge recess or similar aperture (67) of the grip element (12ₐ).

20. The device as claimed in one of claims 1 through 19, which can be used to replace a joint in the two-dimensional plane.

21. The device as claimed in claim 20, which comprises an integrated spring.
